(19) United States
(12) Patent Application Publication
(10) Pub. No.: US 2004/0002655 A1
(43) Pub. Date: Jan. 1, 2004

(54) System and method for improved transducer thermal design using thermo-electric cooling

(75) Inventors: Mirsaid S. Bolorforosh, Portola Valley, CA (US); Worth B. Walters, Cupertino, CA (US); Sevig Ayter, Cupertino, CA (US)

Correspondence Address: Siemens Corporation, Intellectual Property Department, 186 Wood Avenue South, Iselin, NJ, 08830, US

(73) Assignee: Acuson, a Siemens Company

(21) Appl. No.: 10/183,302

(22) Filed: Jun. 27, 2002

(51) Int. Cl. A61B 8/14

(52) U.S. Cl. 600/459

(57) ABSTRACT

An ultrasound transducer assembly having a housing, a transducer array mounted in the housing, and active cooling mechanism positioned adjacent to the transducer array for actively removing heat generated by the array by transport of heat energy from the affected site. The active cooling mechanism comprises a thermo-electric cooler which utilizes active thermal transport to remove heat from the transducer. The thermoelectric cooler may be used alone or in combination with a phase change material or other system to subsequently remove the heat from the thermo-electric cooler. The thermo-electric cooler is coupled with the flex-circuit layers of the transducer to efficiently remove heat generated within the component layers of the transducer.

### BACKGROUND

Medical ultrasound imaging has become a popular means for visualizing and medically diagnosing the condition and health of interior regions of the human body. With this technique an acoustic transducer probe, which is attached to an ultrasound system console via an interconnection cable, is held against the patient's tissue by the sonographer whereupon it emits and receives focused ultrasound waves in a scanning fashion. The scanned ultrasound waves, or ultrasound beams, allow the systematic creation of image slices of the patients internal tissues for display on the ultrasound console. The technique is quick, painless, fairly inexpensive and safe, even for such uses as fetal imaging.

In order to get the best performance from an ultrasound system and its associated transducers it is desirable that the transducers used to emit and receive ultrasonic pulses be capable of operating at the maximum acoustic intensity allowable by the U.S. Food and Drug Administration (FDA). This will help maximize the signal to noise ratio for the given system and transducer, help achieve the best possible acoustic penetration, and ensure that imaging performance is not limited by the inability to emit the full allowable acoustic intensity. Further, this will allow for maximum performance of the various imaging modes such as color flow, Natural Tissue Harmonic Imaging (“NTHI”) and spectral Doppler. In NTHI mode, the transducer is excited at one frequency and receives the acoustic echoes at a second frequency, typically the second harmonic, in order to account for the non-linear propagation of acoustic waves through tissue and the harmonics created thereby. At the same time, there are practical and regulatory limits on the allowable surface temperature that the transducer may attain as it performs its imaging functions. The Underwriters Laboratory (U.L.) Standard #UL544 “Standard for Safety: Medical and Dental Equipment” specifies an upper limit of 41° C. for the transducer portion contacting the patient's skin. In addition, sonographers prefer to grip a transducer case which is comfortably cool, thereby preventing excess perspiration in their hands and a potential to lose their grip on the device. Further, increased internal temperatures may affect the operational characteristics or capabilities of the transducer components, reducing their efficiency and/or operating capabilities. For example, CMOS integrated circuits, which may be utilized as part of the control circuitry in the transducer, operate faster and more efficiently at lower temperatures.

The introduction of Chirp transmit waveforms, Multi-focus (dynamic transmit focus) and high frame rate imaging modes has significantly increased the requirements for transmit power of the transducer. This increase in operating power has necessarily led to an increase in operating temperatures.

Given that it is desirable to be able to operate at the maximum allowable acoustic intensity and also desirable to control the internal transducer operating temperatures as well as the surface temperature distribution of the patient and user-contacting portions of the transducer's surfaces, thermal engineering is a serious consideration during transducer design. There are essentially two possible paths to proceed on with regard to transducer thermal engineering.

The first path makes use of passive cooling mechanisms and involves insuring that the heat that is generated both by the electroacoustic energy conversion process taking place in the transducer's piezoelements and by the acoustic energy passing through and/or into adjacent transducer materials is passively spread out to as large an external transducer surface area as possible. This heat spreading process is typically achieved internal to the transducer by thermal conduction through solid materials and subsequently from the transducer's external case employing natural free convection to the atmosphere. Ideally the external heat-convecting surface area would consist of the entire transducer's external surface area from which free convection cooling to the atmosphere can potentially take place in an unobstructed manner. Transducer manufacturers have thus incorporated various passively conducting heat-spreading plates and members inside the transducer's interior spaces to ensure the spreading of the heat to the entire transducer case surface. Such members work well, however, it is frequently the ability to get the heat out of the electroacoustic elements themselves and into such adjacent internal thermal-sinking structures such as these commonly used spreading plates that provides a significant portion of the probes total thermal dissipation resistance. If this internal thermal path is not a good one it is difficult to spread the heat generated by the piezoelements around the case. If the heat generated by the piezoelements cannot be removed, and effectively coupled and sunk to the entire transducer case area, then the probe surface portion in contact with the patient runs hotter than desired as this probe portion is directly adjacent the piezoelements. Thus, even in the passive strategy, there is concern concerning three key mechanisms: a) removing the heat from the highly localized piezoelement region; b) spreading said heat efficiently to the external case surfaces; and c) allowing for unobstructed natural convection from the warm transducer surfaces.

In any event, using this passive strategy, maximizing the external probe surface area onto which heat spreads in a fairly uniform manner minimizes the peak surface temperature attained anywhere on the probes surface during steady state convection of the probes heat to the ambient. This passive strategy amounts to spreading the heat load around to minimize the impact of the limited ability of free convection to dissipate heat. Its fundamental limitation is that, for most transducers, even if heat is spread uniformly on the external case surfaces, it only takes a few watts of transducer driving power to cause the average transducer surface temperature to become unacceptable either with respect to the patient or the sonographer. In these cases, and particularly for small transducers having small surface areas, one may find that one is unable to operate at the allowable acoustic intensity limit because of excessive temperatures.

FIG. 1 shows a prior-art medical ultrasound transducer 1 in schematic sectional view. Transducer 1 has a typically polymeric external case 2 which is gripped by the sonographer. The top of the transducer (+Y end) can be seen to have the typical acoustic lens 3 which serves to focus the ultrasound beam in the X-Y plane as it passes into the subject patient. Focusing in the Y-Z plane is done via electronic phase delays between the various piezoelements which are arranged on a Z-axis pitch and spacing passing into and out of the paper as is usual for phased array transducers. The bottom or back of the transducer 1 has emanating from it a flexible coaxial cable bundle 4. The cable 4 is shown in broken view at its midpoint to indicate its considerable length, usually on the order of 6 to 12 feet. Where cable 4 exits from the transducer 1, and specifically where it exits from the transducer case 2, can be seen a flexible strain relief 5. Strain reliefs are usually fabricated from a flexible rubber, such as silicone rubber, and they serve to prevent damage to the cable 4 or chemical leakage into the case 2 at the point of cable/case juncture particularly as cable 4 is flexed by the user.

A transducer cable connector 6 can be seen at the termination of the cable 4 (Y end). The connector 6 is usually of a mass-actuated design and has an appropriate rotatable actuation knob 8 for that function. To the right of the transducer's connector 6 are shown in phantom a mating ultrasound system connector 7 mounted on an ultrasound system console 9. To use the transducer the sonographer would plug connector 6 into mating connector 7 (connectors shown unmated) thereby electrically connecting the transducer 1 to the ultrasound system console 9.

In the interior portion of the bottom of transducer 1, inside of polymeric case 2, portions of numerous electrical interconnects 10 (indicated by partial dotted lines) run from the transducer device 1 into the cable 4 and, in turn, into the connector 6. Generally a large number of interconnects 10 comprising coaxial wires of controlled impedance are provided in cable 4 to carry the electrical impulses transmitted to and received from the individual piezoelements making up the phased array. The details of how the interconnects 10 are mated to the piezoelements or to the connector are not shown as it is not critical to the understanding of this invention. It should be generally understood that numerous interconnects 10 pass from the transducer 1 and its piezoelements through the cable to the connector 6 and these serve an electrical function. Interconnects 10 must physically be routed through the interior of the back of the transducer case 2, and around whatever other means, thermal or otherwise, are located therein.

The electroacoustic transducer device assembly 50 is packaged and operated inside the confines of the polymeric case 2. Assembly 50 is shown schematically in FIG. 1 and in FIG. 2. Assembly 50 comprises acoustic backer material 11, a piezoelements 12 and one or more (one shown) acoustic matching layers 13. While the lens 3 is not shown in FIG. 2, it may also be considered part of the Assembly 50. Acoustic backer material 11 serves the functions of attenuating acoustic energy which is directed backwards to minimize reverberations and ringiness, and as a mechanical support for piezoelements 12. Materials used to fabricate backer 11 are generally poorly or only modestly thermally conductive as it is exceedingly difficult to design a highly thermally conductive yet acoustically highly lossy material. Piezoelements 12 may, for example, be fabricated from lead zirconate titanate (PZT) or composite PZT in a manner well-known to one of average skill. On top of piezoelements 12 is the matching layer or layers 13 which serve to act as an acoustic impedance transformer between the high acoustic impedance piezoelements 12 and the low acoustic impedance, human patient. (The human patient is not shown, but it should be understood that the patient is in contact with lens 3.)

The piezoelement material, typically PZT, is a ceramic having generally poor to modest thermal conductivity. The matching layer(s) 13 materials also frequently have poor to modest thermal conductivity because of their conflicting acoustic requirements. It is to be noted that the backer 11, the piezoelements 12 and the matching layer(s) 13 are all intimately bonded to each other and to the lens material 3 such that acoustic energy produced in piezoelements 12 may pass through the layer interfaces in the +Y-direction freely. Of course reflected acoustic echoes from the body may also likewise pass freely in the −Y direction, back into probe 1.

Not shown in FIG. 1 are horizontally running (+−X axis direction) electrodes in any of the interfaces of the type between lens 3 and layer 13, layer 13 and piezoelements 12 or piezoelements 12 and backer 11. Adequate thin electrodes must be present to apply and sense electrical potentials across the top and bottom surfaces of the piezoelements 12. Electrical interconnects 10 are typically routed and connected to such dedicated interface electrodes on a piezoelement by piezoelement basis (connections and routing not shown). The interface or surface electrodes are required to make electrical contact to each piezoelements 12 without appreciably negatively impacting the acoustic performance spectrum of transducer 1. Thus, such electrodes are typically chosen to be very thin, metallic, and have very little mass. This, in turn, causes the electrodes to be poor thermal conductors in the lateral X-direction.

Also shown in FIG. 1 are two symmetrically situated pairs of passive thermal conduction enhancement members 14 and 15 arranged on each side of assembly 50. Thermal member 14 is schematically shown physically and thermally connected to the edge region of element array 12 and layer 13, and possibly also to the ends of the interfacial or surface electrodes (not shown). Thermal member 15 is schematically shown thermally and physically connected to member 14. The members 14 and 15 are arranged to be in close juxtaposition and in good thermal contact with the interior walls of case 2. It will be noted that thermal member 15 may typically be thicker (as shown) and therefore more thermally conductive than member 14 given the increased space toward the cable end of the transducer. In one such representative example, items 14 would consist of thin films of flexible copper, perhaps in the form of a flexible circuit, extending away from the edges of the piezoelement array 12 and possibly emanating from within an interface such as the interface between backer 11 and array 12, array 12 and layer 13 or layer 13 and lens 3 wherein it also serves an aforementioned electrode function. In this example, the primary purpose of member (or flex circuit) 14 is electrical interconnection as necessary in the interfaces between at least certain of the laminated layers. Items 15 would typically consist of aluminum or copper plates, perhaps between 0.010-0.080 inches thick, which are bonded or thermally coupled intimately to the inner surfaces of case 2. The joint between members 14 and 15 must be thermally conductive. If member 14 is an electrical flex circuit used for interconnection, then care would be taken to provide only a thermal joint and not an electrical joint so as not to short out the flex traces which need to be routed (not shown) backwards to interconnects 10.

As the sonographer or user images with transducer probe 1, the system console 9 transmits a series of electrical pulses through the connectors 7,6 and cable 4 to the acoustic array of piezoelements 12. The electroacoustic piezoelements 12 convert the electrical pulses to acoustic output energy emanating from the rubber lens 3 into the patient. During the ultrasound reception portion of the acoustic beamforming, the piezoelement senses in a passive mode the electrical disturbance produced by acoustic energy bounced off of internal patient tissue and reflected back into the transducer 1. It is primarily the transmit portion of imaging when heat is produced by the piezoelements. This is because the electroacoustic energy conversion process is less than 100% efficient. Thus the piezoelements 12 act as unintended heaters. Secondly, as ultrasound energy is produced by the piezoelements 12, it is somewhat absorbed by layers 13 and lens 3, such layers usually not being totally lossless. The unavoidable nonzero portion of acoustic energy which is directed away from the patient into the backer 11 also serves to generate heat in backer 11. Thus, we have heat being directly generated in the piezoelements 12 and indirectly generated in backing material 11, matching layer(s) 13 and lens 3.

A thermal member 14, if comprised of a flexible circuit being formed in part of a thin metal such as copper, offers modest thermal conduction of heat generated by piezoelements 12 laterally in the X direction to the edges of the device and then downward to some more significant thermal sink, such as 15. The purpose of member 15 is to render isothermal the inner surface of the case 2 so that heat may be encouraged to flow across the case wall at all locations. The thermal purpose of member 14 is to get the heat away from the piezoelements 12 and redirected so that it can be flowed into said isothermalization member 15. Using the combination of thermal elements 14 and 15 it has been possible to passively spread the heat out isothermally to most of the interior case 2 surfaces. It should be understood that case 2, being fabricated of a polymer, will typically conduct heat poorly. It is therefore critical to get the heat spread out over most or all of the interior surface of case 2 so that although the thermal resistance across the thickness of the case wall 2 is high, there is considerable surface area to compensate for this fact and keep the overall thermal resistance between the elements and the environment as low as possible.

Heat which is generated in matching layer(s) 13 and lens 3 may also be conducted downward toward the piezoelements 12 or to their interfacial electrodes (not shown) which can, in turn, pass heat to the edges of the stack for redirection downward in the −Y direction via member 14 for example. When transducer probe 1 is in contact with a patient's tissue, some heat may pass directly into the patient. In any event, the U.L. limitation on skin or tissue temperature severely limits the temperature of the lens, and heat dissipation toward the patient.

Heat which is generated in backing material 11 may be passed to thermal means such as member 15. Member 15 may be arranged to actually envelope or wrap around backer material 11 in the form of a metallic thermal container or can (not shown) in order to facilitate the passage of heat from backing material 11 into thermal member 15 and out of transducer 1.

Thus, the ability of probe 1 to shed heat to the environment is governed primarily by passive free convection of heat from the probe's external surfaces. There is a rather limited capacity to remove heat by natural convection of air past the external probe surface even in this optimal isothermalized example. In practice, given the limits on the temperature of lens 3 and sonographer gripping comfort, it is not possible to dissipate more than a few watts of thermal energy in this passive prior-art manner. Also, different sonographers typically cover different amounts of the probe surface with their hands as they grip it, and in some cases much of the heat is being transmitted by conduction directly into the sonographer's hand(s). This can produce sonographer discomfort and a poor grip. If the only heat dissipating surface and path available is the external case surface dissipating by convection to the atmosphere or by conduction into the patient and/or the sonographer's hand, then severe power dissipation limits of a few watts will apply, particularly to small probes having small surface areas even if that surface area is isothermalized.

Others have attempted to increase the lateral (X-axis) and/or vertical (Y-axis) thermal conductivity of acoustic backing material 11, piezoelements 12 and acoustic matching layers 13. Although these measures may help keep the face of the acoustic array more isothermal particularly for very large array probes, they do nothing to increase the capacity to remove heat from the probe's external surfaces in an improved manner.

An extension of the passive-cooling approach has included an attempt to conduct or spread some of the heat down the length of the attached cable in order to permit the cable to offer more passive convection surface area. This helps the situation only incrementally because of the user-preferred small diameter cable and the difficulty of providing much of a thermally conductive path in such a small diameter cable without compromising the desired flexibility and compactness of the cable. Such an incremental measure is described in U.S. Pat. No. 5,213,103 “Apparatus for and method of cooling ultrasonic medical transducers by conductive heat transfer” by Martin, et al.

As a specific example a copper braid could be routed from the case 2 interior into at least some limited length of the cable 4 adjacent to device 1. This copper braided thermal means may be connected to a thermal means in the case such as depicted member 14, 15 or 14 and 15 or may also serve as item 15 for example. This tact essentially creates additional dissipative surface area on the cable.

It should be noted that for endocavity transducers (probes inserted internally into the human body) heat is dissipated both by direct conduction to the patient's internal tissues and fluids, as well as by the conduction out the cable and convection from the exposed transducer handle which remains external to the patient's cavity. We must also control the maximum surface temperatures attained by these probes.

The second strategy for cooling transducers is to utilize active cooling rather than passive cooling in order to dissipate heat well beyond that which can be passively convected or conducted from the external transducer surfaces. Active cooling means that one provides a means to actively remove heat from the transducer such as by employing a pumped coolant or other active refrigeration means. Using active cooling one may ensure that one is always able to operate the acoustic transducer up to the allowable acoustic intensity limit while also maintaining acceptable surface temperatures regardless of how small the transducer is or how much surface area it offers for cooling relative to its acoustic intensity.

At least part of the reason active cooling has not yet been used is because of the apparent cost, reliability and the ease-of-use issues associated with it. There is a well-established continued trend in the ultrasound industry toward reliable “solid-state” phased array transducers with no moving parts and with excellent chemical resistance to disinfection procedures, including procedures involving total chemical immersion for extended periods. There is a more recent trend toward minimizing the cost of ownership for all medical implements as well as any need to service or repair them. Both of these trends place very severe constraints on any potential active transducer cooling means for use in the hospital, clinic or doctor's office environment.

Finally, one must keep in mind that imaging transducers are plugged into and unplugged from the ultrasound console's various connector ports in a varying personalized manner, thus any active cooling scheme should preferably continue to allow for the freedom to do this and should not substantially complicate the integrity or ease of this connection. Large numbers of connector plug/unplug cycles should also not degrade the performance of the active cooling means. Any active cooling scheme should involve minimal additional maintenance and should be as transparent to the user as possible.

### SUMMARY

The present invention is defined by the following claims, and nothing in this section should be taken as a limitation on those claims. By way of introduction, the preferred embodiments described below relate to an ultrasound transducer assembly. The assembly includes a housing, a transducer mounted in the housing, the transducer operable to transmit ultrasonic energy along a path, the transducer comprising a plurality of component layers, each of the component layers separated by a heat conductive layer, and a thermo-electric cooler mounted in the housing and positioned outside of the path, the thermoelectric cooler being thermally coupled with the heat conductive layer for actively removing heat generated by the transducer by active thermal transport of heat energy directly from the heat conductive layer.

The preferred embodiments further relate to a method of cooling an ultrasound transducer. In one embodiment, the method includes providing a transducer mounted in a housing, the transducer operable to transmit ultrasonic energy along a path, the transducer comprising a plurality of component layers, each of the component layers separated by a heat conductive layer, coupling, thermally, a thermoelectric cooler, mounted in the housing and positioned outside of the path, with the heat conductive layer, and removing, actively, heat generated by the transducer by active thermal transport of heat energy directly from the heat conductive layer.

Further aspects and advantages of the invention are discussed below in conjunction with the preferred embodiments.

### BRIEF DESCRIPTION OF THE DRAWINGS

FIG. 1 depicts a partial cross-sectional view of a typical industry-standard, solid-state, phased array transducer with its accompanying cable, system connector, system-console, mating connector, and typical passive heat distribution plates.

FIG. 2 depicts a side view of a piezoelement transducer assembly of the type used in the device shown in FIG. 1.

FIG. 3 is a partial cross-sectional view of a transducer assembly wherein a thermoelectric cooling device is thermally coupled to the heat dissipating piezoelements and their local associated passive heat dissipating members.

FIG. 4 depicts a block diagram of a transducer assembly according to one embodiment.

FIG. 5A shows a micro-mechanical based ultrasound transducer assembly according to an alternate embodiment.

FIG. 5B shows a micro-mechanical based ultrasound transducer assembly according to another alternate embodiment.

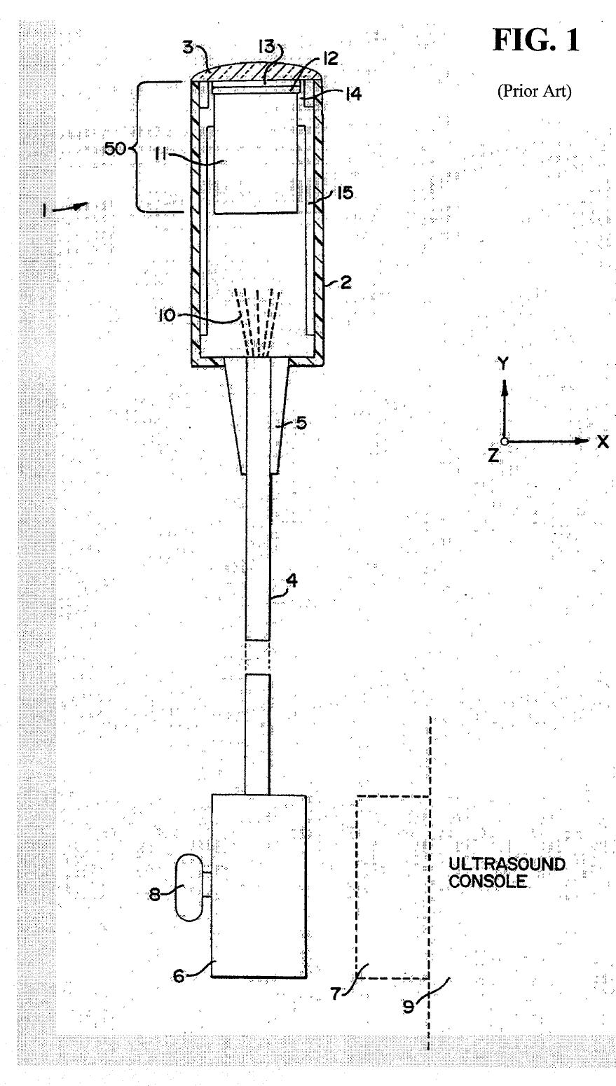

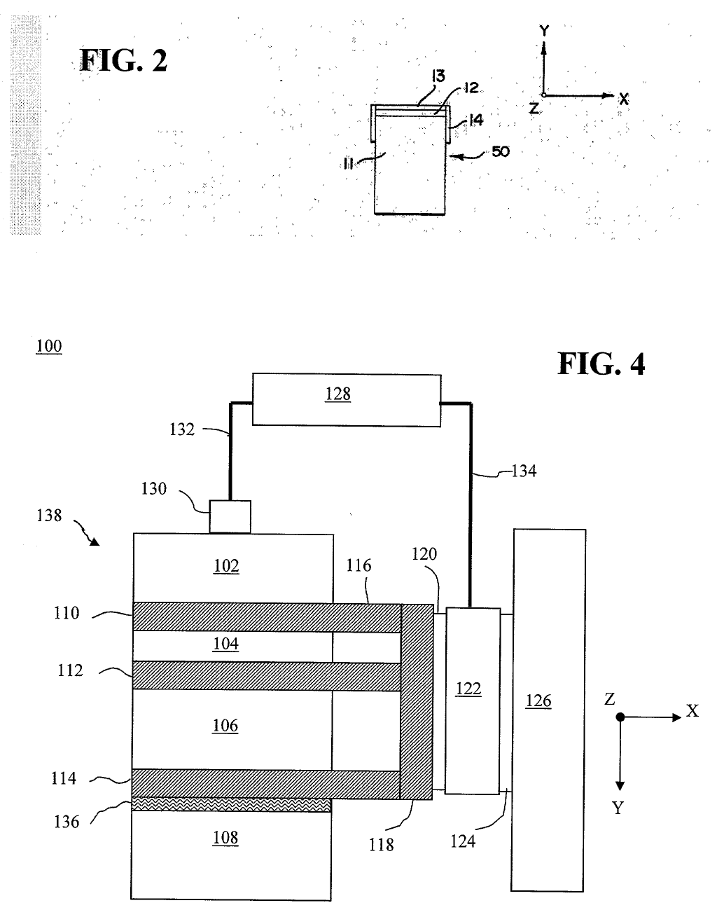

### DETAILED DESCRIPTION OF THE PRESENTLY PREFERRED EMBODIMENTS

The invention relates to imaging of materials in living tissue, and in particular to increasing the power output of transducing means used for acoustic imaging by compensating for thermal problems associated with such increased power output using active cooling. For more information about using active cooling in transducers, refer to U.S. Pat. No. 5,560,362, herein incorporated by reference.

As described above, current transducer designs incorporate a flexible circuit board connection to one side of the piezoelectric material for signal connection, and a copper foil acting as the ground electrode at the other side of the piezoelectric material. Some of the heat generated by the PZT and the first impedance matching layer can be taken away form the face of the probe using these layers. The current transducer designs thermally connect these copper layers to the thermal dissipation plates at the transducer handle, which results in improved thermal performance.

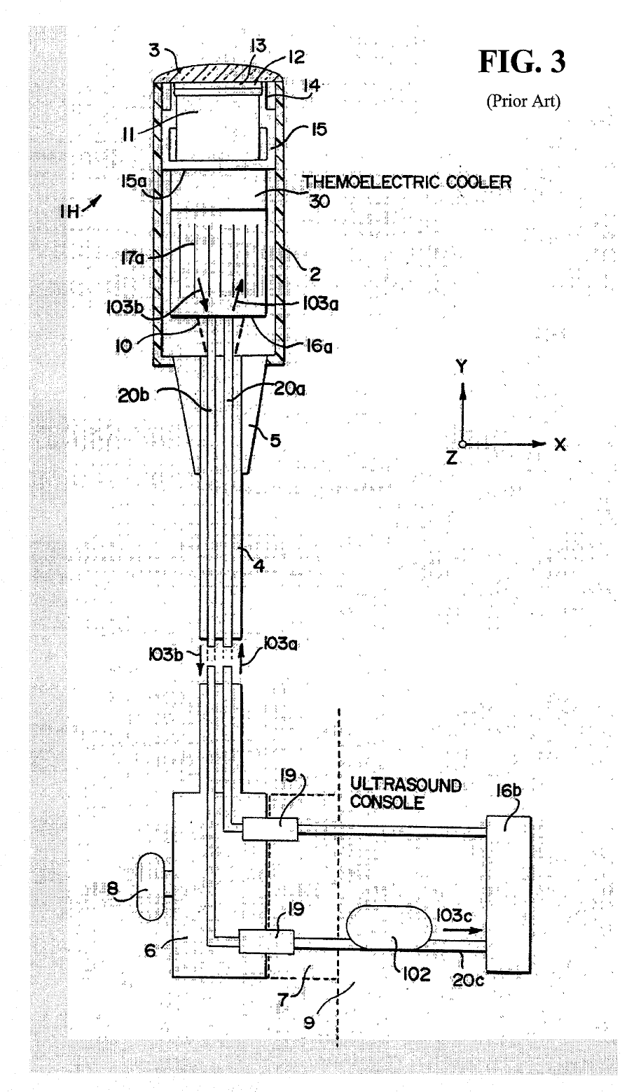

FIG. 3 depicts a system which uses a thermoelectric cooling device 30, such as a Peltier device, in the thermal path between passive conducting members 15, 15A and heat exchanger 16A. Note that the thermal member 15A extends beneath acoustic backer 11 such that heat may be deposited in the top cooling surface (+Y surface) of thermoelectric cooler 30. Cooler 30 would typically consist of an electrically powered junction device capable of establishing a thermal gradient and transporting heat through its thickness along the Y axis (sometimes referred to as a Peltier device). Such devices, although capable of moving appreciable quantities of heat, are typically rather inefficient. Thus, the cooling system components described in U.S. Pat. No. 5,560,362 may be used to carry away not only the piezoelement heat pumped by cooler 30, but also the waste heat generated by the cooler 30 itself. Specifically, such thermoelectric coolers 30 are available, for example, from Marlow Industries, Inc. (10451 Vista Park Road, Dallas, Tex. 75238) with heat removal capacities covering the range from 1 to 150 watts. The cold (cooling) side of cooler 30 may, depending on the heat load and specific type of cooler, have the capacity to subcool between 10 and 100 degrees centigrade. Use of a thermoelectric cooler 30 offers advantages of dynamic real-time temperature control of the transducer piezoelements and/or the thermal capacity to actually subcool the piezoelements 12 as described without requiring a conventional freon-style refrigeration system. The reader will realize that the thermoelectric cooler 30 may be arranged to dump its heat to any of the other known thermal dissipation means.

A specific advantage of a thermoelectric cooler 30 is appreciated when performing high frequency ultrasound imaging of near-surface tissues. In these growing applications, increasing amounts of heat energy are being generated in the probe and in the tissue as manufacturers attempt to achieve the highest possible resolution at the maximum allowable acoustic intensities. It would be rather difficult to maintain a reasonable lens temperature unless a cooling device 30 having very large cooling capacity (a device capable of subcooling may serve this purpose) is present in close proximity to the piezoelements, lens and tissue.

As opposed to coupling the thermoelectric cooler with the passive conducting members 15, 15A as described above, one can improve the heat transfer between the copper foils and the thermal plates by placing the thermoelectric cooling device in between, such that the hot junction of the thermo-electric cooling device is in contact with the thermal plates and the cold junction is in contact with the copper foil. Herein, the phrase “coupled with” is defined to mean directly connected to or indirectly connected through one or more intermediate components. This helps to maintain the temperature of the copper foil at a lower temperature while increasing the temperature of the thermal plates. The thermal plates are in contact with the plastic parts at the transducer handle. If the transducer handle is made of thermally conductive materials, the overall thermal dissipation of the device may be improved. Alternatively, the thermal plates or the hot junction of the thermo-electric cooling device can be connected to the overall transducer cable jacket. The overall transducer cable shield has a large surface area. The surface area of the conductors in the cable jacket shield is 200 cm×1 cm. This is much larger than the surface area of the transducer (1.9 cm×1.4 cm) or the handle (about 80 cm²).

FIG. 4 shows a block diagram of a transducer assembly 138 according to a first embodiment. The assembly 138 includes a backing layer 102, a piezo-electric layer 104, impedance matching layer 106, and a mechanical lens 108. The piezo-electric layer 104 is preferably a PZT layer 104, as described above, and the mechanical lens 108 is preferably made of silicone rubber, although one of ordinary skill in the art will recognize that other materials may be used. Further, one of ordinary skill in the art will appreciate that other mechanisms for generating ultrasonic energy may also be used as will be discussed below.

In the first embodiment, flex circuit layers 110, 112 including flexible signal connections and electrical ground connections, are sandwiched between the transducer layers 102, 104, 106. It will be appreciated that the transducer assembly may have more or fewer functional and electrical connectivity layers and that other materials may be used in place of or in addition to the disclosed materials. The Flex circuit layers 110, 112 preferably comprise a material that is thermally conductive in addition to being electrically conductive, such as copper. A thermo-electric cooler 122, and specifically, the cold junction of the thermo-electric cooler 122, is thermally coupled 116, 118, 120 with the flex circuit layers 110, 112. The thermal coupling is preferably implemented so as not to interfere with the electrical operation of the flex circuit layers 110, 112 and operation of the transducer 138. The thermoelectric cooler 122, and specifically, the hot junction of the thermo-electric cooler 122, is thermally coupled with a heat sinking device 126. The heat sinking device 126 may be an active or passive cooling system as described above, the transducer case, or a phase-change material based heat dissipation system, as described below. The heat sinking device 126 removes heat dissipated by the thermo-electric cooler 122 from the transducer 138 as well as heat generated by operation of the thermo-electric cooler 122 itself.

In operation of the transducer 138, heat is generated within the various layers 102, 104, 106, 108 as described above. The generated heat is convected away from the layers 102, 104, 106, 108 by the heat conductive flex circuit layers 110, 112 and out of the transducer along the thermal path 116, 118, 120 to the thermoelectric cooler 122. An electrical current passing through the thermo-electric cooler (electrical connections not shown) causes the thermo-electric cooler 122 to convect heat from its cold junction to its hot junction, as described above and as is known in the art. The generated heat is then passed to the heat sinking device 126. By coupling the thermoelectric cooler 122 directly to the flex-circuit layers 110, 112, the heat generated within the layers 102, 104, 106, 108 of the transducer 138 is more effectively dissipated. As noted above, it is frequently the ability to get the heat out of the electroacoustic elements themselves and into adjacent internal thermal-sinking structures that provides a significant portion of the probes total thermal dissipation resistance. Given that the flex circuit layers 110, 112 are typically poor thermal conductors, as described above, this placement of the thermoelectric cooler 122 substantially proximate to the transducer assembly 138 and in direct thermal contact with the flex circuit layers 110, 112 without the need for intermediary passive thermal members, results in more effective and efficient heat dissipation.

In a second embodiment, the heat generated during operation of the transducer 138 can also be taken away from the other layers 102, 104, 106, 108 of the transducer assembly 138 such as the mechanical lens/window 108 or the impedance matching layers 106. To accomplish this, b a thin (<0.1 λ, λ being the wavelength in the layer material) layer of thermally conductive material, such as copper or a metal mesh, may be embedded in the RTV lens 108 and/or impedance matching layers 106 and similarly coupled with the thermoelectric cooler 122.

As was noted, the cooling capacity of the thermo-electric device 122 can be controlled via the input electrical current to the device 122. In a third embodiment, a feed back control circuit 128 is provided to monitor the temperature of the probe and adjust the electrical current supplied to the thermo-electric cooling device 122 in order to maintain the optimum condition under all operating environments. The feed back control circuit 128 is coupled 134 with the current supply control of the thermoelectric cooler 122 and with a temperature sensor 130 which allows the circuit 128 to monitor the probe temperature. The feed back control circuit 128 may be controlled by the user or controlled automatically to maintain desired probe operating temperatures, indicate or prevent thermal overloads, or otherwise maintain optimal probe operation. Further, the feed-back control circuit 128 may be used to efficiently operate the thermo-electric cooler 122 only when necessary to achieve a desired probe temperature thereby avoiding unnecessary operation of the cooler 122.

In a fourth embodiment, the heat sinking device 126 includes a phase change material such as wax in the case or case walls of the transducer housing (not shown) to dissipate the heat generated by the thermo-electric cooling device 122 itself. The polarity of the voltage supplied to the cooler 122 may be reversed when the transducer 138 is not generating heat or in not operating in a thermally limited mode to cool down the phase change material. This may be controlled by the feed back control circuit 128.

In a fifth embodiment, separate thermo-electric coolers 122 may be provided to dissipate the heat generated by one or more of the layers 102, 104, 106, 108, as described above.

It will also immediately be recognized by those skilled in the art that one may easily use the thermo-electric cooler 122 to also heat the probe such that it is warm and comfortable to the patient's touch when first used. Alternatively one might ensure that the probe operates at all times at a desired temperature setpoint (including when the probe is first switched on) or below such a setpoint or above a lower setpoint and below a second higher setpoint. This can be achieved by reversing the polarity of the current supplied to the thermo-electric cooler 122 as described above. The cooler 122 might also be used to cool the probe to prevent damaging it during hot disinfection or sterilization procedures used to clean the probe.

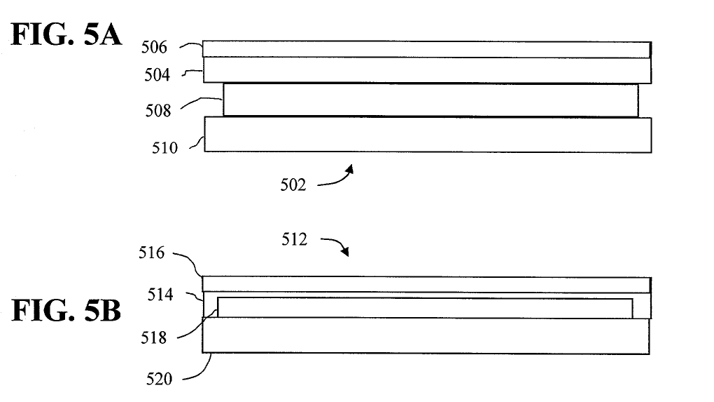

FIGS. 5A and 5B show cross sectional views of a sixth embodiment using micro-mechanical based ultrasound transducers such as capacitive micro-mechanical ultrasound transducers (“cMUT's”). Such transducers use micro-mechanical components fabricated using integrated circuit fabrication techniques to generate the ultrasonic energy and receive the resultant echoes for diagnostic imaging. For more information about cMUT's and other micro-mechanical based ultrasound transducers, refer to U.S. Pat. No. ______ “DIAGNOSTIC MEDICAL ULTRASOUND SYSTEMS AND TRANSDUCERS UTILIZING MICRO-MECHANICAL COMPONENTS”, formerly U.S. patent application Ser. No. 09/223,257, herein incorporated by reference.

FIG. 5A shows a micro-mechanical based ultrasound transducer assembly 502 having a thermo-electric cooling device 508, as described above, attached thereto. The micro-mechanical transducer elements 506 are fabricated on a substrate 504, such as a silicon wafer, although other substrate materials may be used. The cold junction of the thermoelectric cooling device 508 is thermally coupled, such as by a thermally conductive glue, with the back of the substrate 504 so as to conduct heat generated by the micro-mechanical transducer elements 506 away from the substrate. A heat sinking device 510, such as the heat sinking devices described above, is coupled with the hot junction of the thermo-electric cooling device 508 to dissipate the heat generated therefrom.

FIG. 5B shows a transducer assembly 512 according to an alternate embodiment. The transducer assembly 512 includes micro-mechanical transducer elements 516 fabricated on a substrate 514, such as a silicon wafer. A thermo-electric cooler 518 is fabricated on the opposite side of the substrate material 514 such that the cold junction of the thermo-electric cooler 518 is proximate to the micro-mechanical transducer elements 516. A heat sinking device 520, such as the heat sinking devices described above, is coupled with the hot junction of the thermo-electric cooling device 518 to dissipate the heat generated therefrom. Alternatively, the thermoelectric cooler 518 can be integrated/fabricated on the same side of the substrate 514 as the micro-mechanical ultrasound elements 516, for example, off to one side.

It will be appreciated that the embodiments utilizing micro-mechanical ultrasound elements may use the feed-back control circuit described above. Further, the heat sinking devices 510, 520 may be active or passive devices, as described above, and appropriately designed to channel the dissipated heat to a desired point within or outside the transducer housing.

It is therefore intended that the foregoing detailed description be regarded as illustrative rather than limiting, and that it be understood that it is the following claims, including all equivalents, that are intended to define the spirit and scope of this invention.

We claim:

1. An ultrasound transducer assembly, comprising:
a housing;
a transducer mounted in said housing, said transducer operable to transmit ultrasonic energy along a path, said transducer comprising a plurality of component layers, each of said component layers separated by a heat conductive layer; and
a thermoelectric cooler mounted in said housing and positioned outside of said path, said thermoelectric cooler being thermally coupled with said heat conductive layer for actively removing heat generated by said transducer by active thermal transport of heat energy directly from said heat conductive layer.

2. The ultrasound transducer assembly of claim 1, wherein said plurality of component layers comprise a backing layer, a PZT layer, and an impedance matching layer, wherein said PZT layer is between said backing and said impedance matching layers, said heat conductive layer comprising first and second heat conductive layers, said first heat conductive layer located between said backing and said PZT layers, and said second heat conductive layer located between said PZT and said impedance matching layers.

3. The ultrasound transducer assembly of claim 2, wherein said plurality of layers further comprise a lens layer, said impedance matching layer being between said PZT layer and said lens layer, said heat conductive layer comprising a third heat conductive layer located between said impedance matching and lens layers.

4. The ultrasound transducer assembly of claim 3, wherein said lens layer further incorporates a thermally conductive material coupled with said third heat conductive layer.

5. The ultrasound transducer assembly of claim 1, wherein said heat conductive layer is electrically conductive.

6. The ultrasound transducer assembly of claim 5, wherein said heat conductor layer carries control signals to said transducer and carries response signals from said transducer.

7. The ultrasound transducer assembly of claim 1, wherein said heat conductive layer comprises a flex-circuit.

8. The ultrasound transducer assembly of claim 1, wherein said thermo-electric cooler is located substantially proximate to said transducer.

9. The ultrasound transducer assembly of claim 1, wherein said thermoelectric cooler has a thermal capacity range sufficient to cool said transducer below ambient temperature when said transducer is operating.

10. The ultrasound transducer assembly of claim 1, wherein said thermo-electric cooler comprises a Peltier device.

11. The ultrasound transducer assembly of claim 1, further comprising a feed-back circuit coupled with said transducer, said feed-back circuit comprising a temperature sensor operative to sense an operating temperature of said transducer and a control circuit, coupled with said thermoelectric cooler and operative to control said thermo-electric cooler in response to said sensed operating temperature.

12. The ultrasound transducer assembly of claim 11, wherein said feed-back circuit is further operative to selectively maintain said operating temperature at a predefined threshold.

13. The ultrasound transducer assembly of claim 12, wherein said feed-back circuit is further operative to maintain said operating temperature while minimizing power consumption of said thermo-electric cooler.

14. The ultrasound transducer assembly of claim 11, wherein said feed back circuit is further operative to monitor an efficiency of said thermo-electric cooler.

15. The ultrasound transducer assembly of claim 1, wherein said thermo-electric cooler is further thermally coupled with a phase change material characterized by a capability to absorb heat from said thermo-electric cooler through a change from a first phase to a second phase.

16. The ultrasound transducer assembly of claim 15, wherein said phase change material comprises wax.

17. The ultrasound transducer assembly of claim 15, wherein said thermo-electric cooler is further operative to be operated in reverse to dissipate heat from said phase change material.

18. A method of cooling an ultrasound transducer, comprising:
providing a transducer mounted in a housing, said transducer operable to transmit ultrasonic energy along a path, said transducer comprising a plurality of component layers, each of said component layers separated by a heat conductive layer;
coupling, thermally, a thermo-electric cooler, mounted in said housing and positioned outside of said path, with said heat conductive layer; and
removing, actively, heat generated by said transducer by active thermal transport of heat energy directly from said heat conductive layer.

19. The method of claim 18, wherein said plurality of component layers comprise a backing layer, a PZT layer, and an impedance matching layer, wherein said PZT layer is between said backing and said impedance matching layers, said heat conductive layer comprising first and second heat conductive layer, said method further comprising:
locating said first heat conductive layer between said backing and said PZT layers, and locating said second heat conductive layer between said PZT and said impedance matching layers.

20. The method of claim 19, wherein said plurality of layers further comprise a lens layer, said impedance matching layer being between said PZT layer and said lens layer, said heat conductive layer comprising a third heat conductive layer, said method further comprising:
locating said third heat conductive layer between said impedance matching and lens layers.

21. The method of claim 20, further comprising:
incorporating a thermally conductive material with said lens layer and coupling said thermally conductive material with said third heat conductive layer.

22. The method of claim 18, wherein said heat conductive layer is electrically conductive.

23. The method of claim 22, further comprising:
communicating control signals to said transducer over said heat conductive layer; and
communicating response signals from said transducer over said heat conductive layer.

24. The method of claim 18, wherein said heat conductive layer comprises a flex-circuit.

25. The method of claim 18, further comprising:
locating said thermo-electric cooler substantially proximate to said transducer.

26. The method of claim 18, further comprising:
cooling said transducer below ambient temperature using said thermoelectric cooler when said transducer is operating.

27. The method of claim 18, wherein said thermoelectric cooler comprises a Peltier device.

28. The method of claim 18, further comprising sensing an operating temperature of said transducer; and
controlling said thermoelectric cooler based on said sensing.

29. The method of claim 28, wherein said controlling further comprises:
maintaining, selectively, said operating temperature at a pre-defined threshold.

30. The method of claim 29, wherein said controlling further comprises:
minimizing power consumption of said thermoelectric cooler while maintaining said operating temperature.

31. The method of claim 28, further comprising:
monitoring efficiency of said thermo-electric cooler.

32. The method of claim 18, further comprising:
absorbing said heat from said thermo-electric cooler using a phase change material characterized by a capability to absorb heat from said thermoelectric cooler through a change from a first phase to a second phase.

33. The method of claim 32, wherein said phase change material comprises wax.

34. The method of claim 32, further comprising:
operating said thermo-electric cooler in reverse to dissipate heat from said phase change material.

35. A ultrasound transducer assembly comprising:
a transducer means mounted in a housing, said transducer means for transmitting ultrasonic energy along a path, said transducer means comprising a plurality of component layers, each of said component layers separated by a heat conductive layer means; and
a thermo-electric cooling means for removing, actively, heat generated by said transducer by active thermal transport of heat energy directly from said heat conductive layer, said thermo-electric cooling means being mounted in said housing and positioned outside of said path, the thermo-electric cooling means coupled with said heat conductive layer means.

36. An ultrasound transducer assembly, comprising:
a housing;
a transducer mounted in said housing, said transducer operable to transmit ultrasonic energy along a path, said transducer comprising a substrate, said substrate having at least one micro-mechanical ultrasound element thereon; and
a thermo-electric cooler mounted in said housing and positioned outside of said path, said thermo-electric cooler being thermally coupled with said substrate for actively removing heat generated by said transducer by active thermal transport of heat energy directly from said substrate.

37. The ultrasound transducer assembly of claim 36, wherein:
said at least one micro-mechanical ultrasound element is fabricated on a first side of said substrate and said thermo-electric cooler is coupled with a second side of said substrate opposite said first side.

38. The ultrasound transducer assembly of claim 36, wherein said thermo-electric cooler is fabricated on said substrate.
